(19) United States
(12) Patent Application Publication
(10) Pub. No.: US 2002/0103534 A1
(43) Pub. Date: Aug. 1, 2002

(54) Flexible transmyocardial implant

(76) Inventors: Guy P. Vanney, Blaine, MN (US); David H. Mowry, Waconia, MN (US)

Correspondence Address: MERCHANT & GOULD PC, P.O. BOX 2903, MINNEAPOLIS, MN, 55402-0903, US

(21) Appl. No.: 09/975,740

(22) Filed: Oct. 10, 2001

Related U.S. Application Data
(60) Provisional application No. 60/304,208, filed on Oct. 11, 2000.

(51) Int. Cl. A61F 2/06; A61M 1/10

(52) U.S. Cl. 623/3.1; 606/153; 623/1.24

(57) ABSTRACT

A transmyocardial implant includes a hollow rigid conduit adapted to be inserted into and retained within the heart wall of a heart chamber. The rigid conduit is sufficiently rigid to withstand collapsing in response to contraction forces of the heart wall. A synthetic flexible conduit has a first end secured to the conduit. The flexible conduit is blood compatible. A second end of the flexible conduit is secured to the coronary vessel. The rigid conduit and the flexible conduit define a blood flow path from the heart chamber to the coronary vessel. The flexible conduit is bonded to and wrapped around the rigid conduit for the blood flow path to be defined by an uninterrupted surface of the flexible conduit.

### CROSS-REFERENCE TO RELATED APPLICATIONS

This application claims priority to U.S. application Ser. No. 09/686,245 filed Oct. 11, 2000 and later converted to U.S. Provisional Application Serial No. 60/304,208.

### I.

### BACKGROUND OF THE INVENTION

1. Field of the Invention

This invention pertains to an implant for passing blood flow directly between a chamber of the heart and a coronary vessel. More particularly, this invention pertains to a flexible transmyocardial implant.

2. Description of the Prior Art

U.S. Pat. No. 5,944,019 issued Aug. 31, 1999 teaches an implant for defining a blood flow conduit directly from a chamber of the heart to a lumen of a coronary vessel. An embodiment disclosed in the aforementioned patent teaches an L-shaped implant in the form of a rigid conduit having one leg sized to be received within a lumen of a coronary artery and a second leg sized to pass through the myocardium and extend into the left ventricle of the heart. As disclosed in the above-referenced patent, the conduit is rigid and remains open for blood flow to pass through the conduit during both systole and diastole. The conduit penetrates into the left ventricle in order to prevent tissue growth and occlusions over an opening of the conduit.

U.S. Pat. No. 5,984,956 issued Nov. 16, 1999 teaches an implant with an enhanced fixation structure. The enhanced fixation structure includes a fabric surrounding at least a portion of the conduit to facilitate tissue growth on the exterior of the implant. U.S. Pat. No. 6,029,672 issued Feb. 29, 2000 teaches procedures and tools for placing a conduit.

Implants such as those shown in the aforementioned patents include a portion to be connected to a coronary vessel and a portion to be placed within the myocardium. Most of the implants disclosed in the above-mentioned applications are rigid structures. Being rigid, the implants are restricted in use. For example, an occluded site may not be positioned on the heart in close proximity to a heart chamber containing oxygenated blood. To access such a site with a rigid, titanium implant, a very long implant must be used. A long implant results in a long pathway in which blood will be in contact with the material of the implant. With non-biological materials, such as titanium, a long residence time of blood against such materials increases the probability of thrombus. The risk can be reduced with anti-thrombotic coatings. Moreover, a rigid implant can be difficult to place while achieving desired alignment of the implant with the vessel. A flexible implant will enhance placement of the implant. U.S. Pat. No. 5,944,019 shows a flexible implant in FIG. 22 of the '019 patent by showing a cylindrical rigid member in the heart wall and a T-shaped rigid member in the coronary artery. The cylindrical and T-shaped rigid members are joined by flexible conduit. Unfortunately, flexible materials tend to be non-biostable and trombogenic and may collapse due to contraction of the heart during systole. PCT/US99/01012 shows a flexible transmyocardial conduit in the form of a cylindrical rigid member in the heart wall and a natural vessel (artery or vein segment) connecting the rigid member to an occluded artery. PCT/US99/00593 (International Publication No. WO99/38459) also shows a flexible conduit. PCT/US97/14801 (International Publication No. WO 98/08456) shows (in FIG. 8c) a transmyocardial stent with a covering of expanded polytetrafluoroethylene.

### III.

### SUMMARY OF THE INVENTION

According to a preferred embodiment of the present invention, a transmyocardial implant is disclosed for establishing a blood flow path through a myocardium between a heart chamber and a lumen of a coronary vessel residing on an exterior of the heart. The implant includes a hollow rigid conduit adapted to be inserted into and retained within the heart wall of a heart chamber. The rigid conduit is sufficiently rigid to withstand collapsing in response to contraction forces of the heart wall. A synthetic flexible conduit has a first end secured to the rigid conduit. The flexible conduit is blood compatible. A second end of the flexible conduit is secured to the coronary vessel. The rigid conduit and the flexible conduit define a blood flow path from the heart chamber to the coronary vessel. The flexible conduit is bonded to and wrapped around the rigid conduit for the blood flow path to be defined by an uninterrupted surface of the flexible conduit.

### IV.

### BRIEF DESCRIPTION OF THE DRAWINGS

FIG. 1 is a side sectional view of an implant according to the present invention;

FIG. 2 is a side sectional view of an implant according to the present invention shown in place in a human heart wall with the implant establishing a direct blood flow path from a heart chamber to a coronary vessel;

FIG. 3 is a perspective view of the implant of FIG. 1;

FIG. 4 is a perspective view of a novel attachment member for attachment to a vessel in lieu of a conventional anastomosis;

FIG. 5 is the view of FIG. 4 shown attached to a vessel;

FIG. 6 is a side sectional view of a tube prior to formation of the attachment member of FIG. 4;

FIG. 7 is a side elevation view of the tube of FIG. 6 with phantom lines indicating a manner of formation of the attachment member of FIG. 4;

FIG. 8 is a side elevation view of the attachment member following the formation of FIG. 7;

FIG. 9 is a top plan view of the attachment member of FIG. 8;

FIG. 10 is the view of FIG. 8 with an optional sewing cuff; and

FIG. 11 is the view of FIG. 8 with an alternative embodiment of the attachment member showing an open cell mesh construction in the vessel.

### V.

### DESCRIPTION OF THE PREFERRED EMBODIMENT

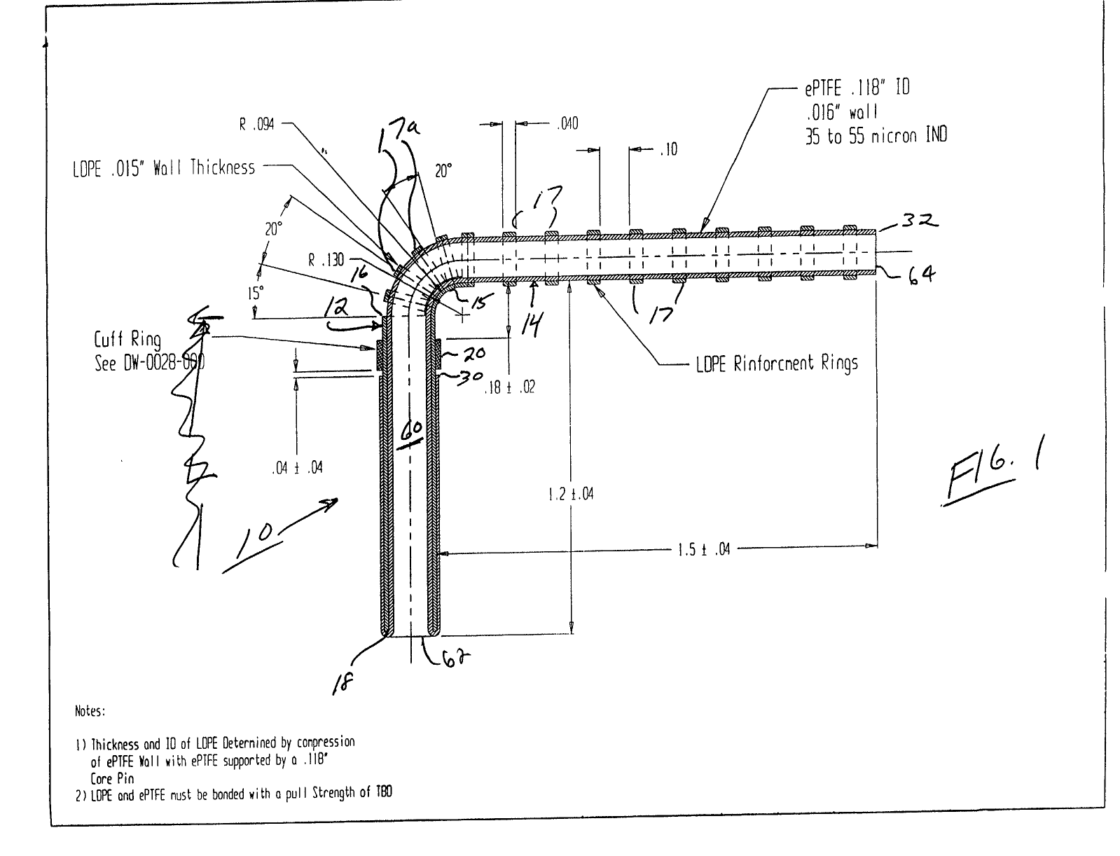

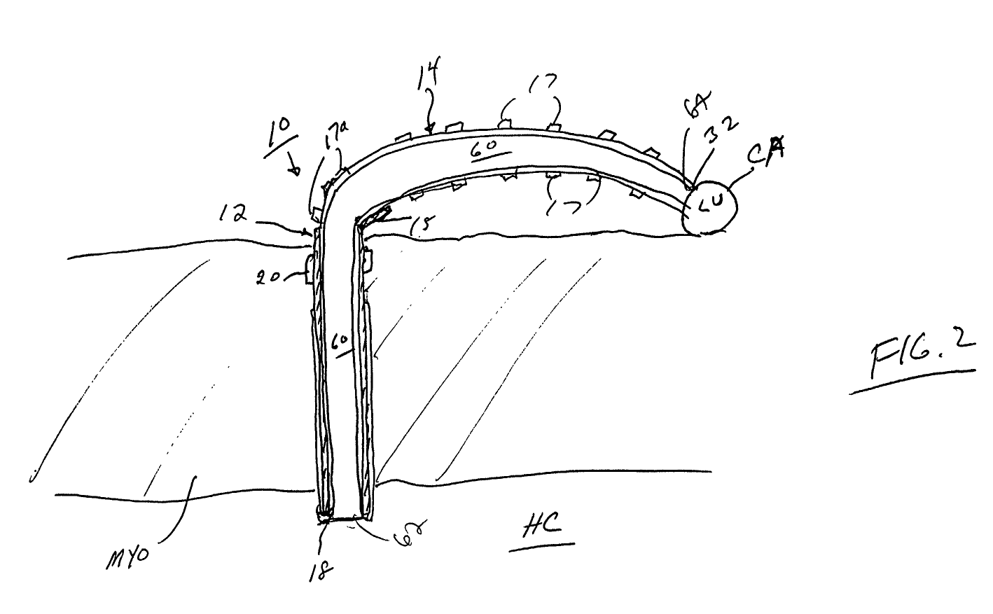

With initial reference to FIGS. 1-3, an implant 10 is shown including a composite of a hollow, rigid cylindrical conduit 12 and a flexible conduit 14. The conduit 12 may be formed of any suitable material. In a preferred embodiment conduit 12 is formed of low density polyethylene (“LDPE”). The material of the conduit 12 is preferably a rigid material in order to withstand contraction forces of the myocardium and hold open a path through the myocardium during both systole and diastole.

The conduit 12 is sized to extend through the myocardium MYO of the human heart to project into the interior of a heart chamber HC (preferably, the left ventricle) by a distance of about 5 mm. The conduit 12 extends from a first (or upper) end 16 to a second (or lower) end 18 (FIG. 1).

As discussed more fully in the afore-mentioned U.S. Pat. No. 5,984,956, the conduit 12 may be provided with tissue-growth inducing material 20 adjacent the upper end 16 to immobilize the conduit 12 within the myocardium MYO. The material 20 surrounds the exterior of the conduit 12 and may be a polyester woven sleeve or sintered metal to define pores into which tissue growth from the myocardium MYO may occur.

The flexible conduit 14 has first and second ends 30, 32 (FIG. 1). The first end 30 of the flexible conduit 14 is inserted through the interior of the conduit 12. The first end 30 is wrapped around the lower end 18 of the conduit 12 such that the first end 30 of the graft 14 covers the exterior of the conduit 12 adjacent the lower end 18 of the conduit 12. The first end 30 terminates spaced from the upper end 16 to expose the tissue-growth inducing material 20.

The first end 30 of the flexible conduit 14 is secured to the rigid conduit 12 by heat bonding along all surfaces of opposing material of the rigid conduit 12 and the flexible conduit 14. At elevated temperatures, the material of the rigid conduit 12 flows into the micro-pores of the material of the flexible conduit 14. The rigid material has a lower melting point than the flexible material.

The rigid conduit 12 and attached flexible conduit 14 are placed in the myocardium MYO with the lower end 18 protruding into the left ventricle HC. The implant 10 thus defines an open blood flow path 60 having a first end 62 in blood flow communication with the left ventricle 82. A second end 64 of the blood flow path 60 communicates directly with the lumen LU of the coronary vessel CA lying at an exterior of the heart wall MYO. To bypass an obstruction in a coronary artery, the end 32 of the flexible conduit 14 is attached to the artery in any suitable manner. For example, the end 32 may be anastomosed to the artery 32 with sutures (not shown) in an end-to-side anastomosis as is done in conventional coronary artery bypass procedures. The end 32 is secured to the artery CA distal to the obstruction.

With the above-described embodiment, the implant 10 permits revascularization from the left ventricle HC to a coronary vessel such as a coronary artery CA (or a coronary vein in the event of a retrograde profusion procedure). The use of an elongated, flexible conduit 14 permits revascularization where the vessel CA is not necessarily in overlying relation to the chamber HC. For example, the implant 10 permits direct blood flow between the left ventricle HC and a vessel CA overlying the right ventricle (not shown). The use of a PTFE flexible conduit 14 results in blood flowing through path 60 being exposed only to PTFE which is a material already used as a synthetic vessel with proven blood and tissue compatibility thereby reducing risk of thrombosis and encouraging endotheliazation. As shown in FIG. 1, the graft 14 is wrapped around the conduit 12 so that no portion of the rigid conduit 12 is in contact with blood within the left ventricle HC.

An interior radius 15 (FIG. 1) is provided on a side of the rigid conduit 12 at end 16. The radius 15 provides support for the flexible conduit 14 and pre-forms the flexible conduit at a preferred 90° bend (a bend of differing degree or no bend could be used).

A plurality of discrete rigid rings 17 are provided along the length of the flexible conduit not otherwise opposing the rigid conduit. Preferably, the rings are LDPE each having an interior surface heat bonded to an exterior surface of the flexible conduit 14. At the radius 15, LDPE rings 17a are integrally formed with the radius 15 with the cross-sectional planes of the rings 17a set at a fixed angle of separation (e.g., about 20 degrees) to support the flexible conduit throughout the 90 degree bend. Again, an interior surface of rings 17a is heat bonded to an exterior surface of the flexible conduit. The rings 17, 17a provide crush resistance. Between the rings 17, 17a, the flexible conduit may flex inwardly and outwardly to better simulate the natural compliance of a natural blood vessel. By way of a further non-limiting example, the discrete rings 17 could be replaced with a continuous helix.

With the foregoing design, an implant of accepted implant material (i.e., LDPE and ePTFE) is formed with blood only exposed to the higher blood compatibility of ePTFE. The constantly open geometry permits a smaller internal diameter of the ePTFE previously attainable with conventional grafts.

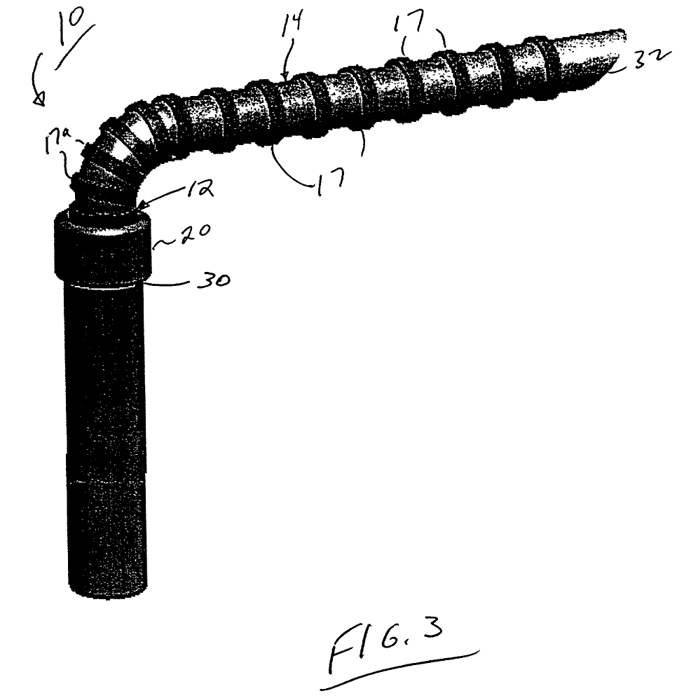

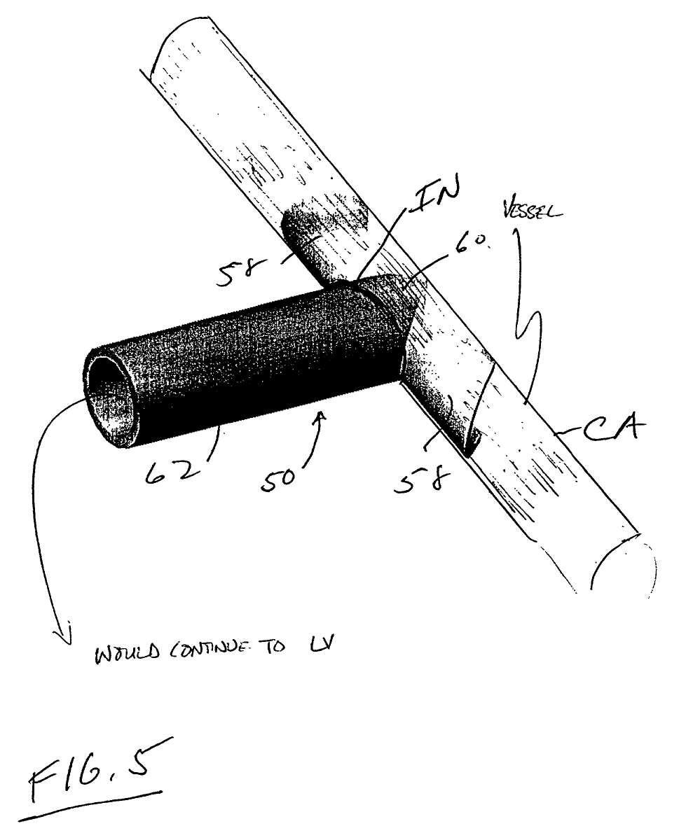

FIGS. 4-11 illustrate an invention for attaching a conduit to a vessel in other than a traditional end-to-side anastomosis while permitting blood to flow from the conduit and in opposite directions with a vessel. The embodiment of the invention is illustrated with respect to use with the conduit 10 of FIG. 1 but may be used with any suitable conduit or graft material.

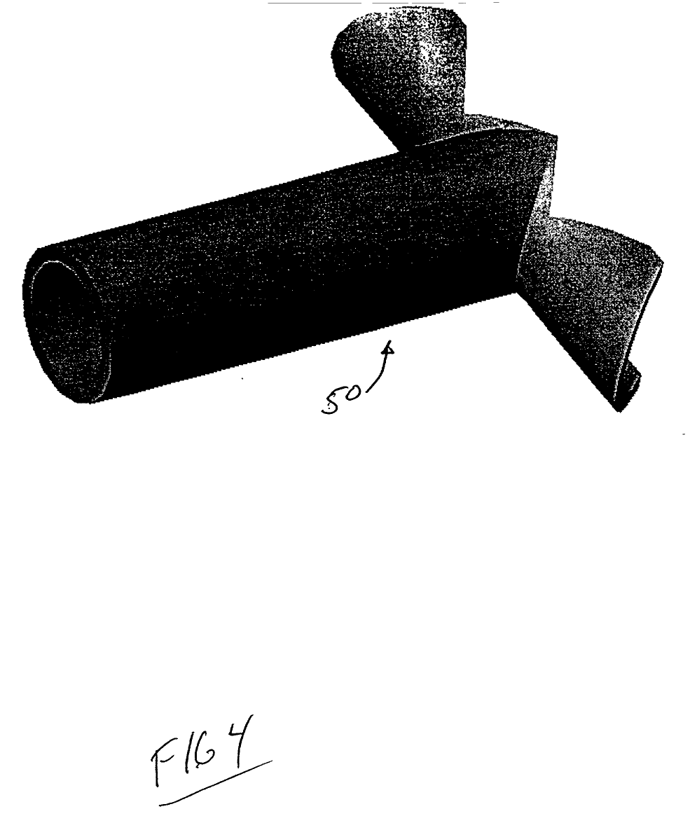

The invention utilizes an attachment member 50 having a generally T-shaped configuration. In a preferred embodiment, the member is formed from a tube 52 of LDPE (FIG. 6) which has interior and exterior lining 54 of ePTFE as described above. In the flexible conduit embodiment described above, the PTFE of the attachment member 50 is an extension of the flexible conduit 14.

The free end 55 of the tube is cut with cuts 56 formed from the center of the free end and angling outwardly to (but not through) the sidewalls of the tube. So cut, two anchor wings 58 are formed on opposite sides of centrally positioned triangular portion 60. The triangular portion 60 is aligned with a cylindrical conduit portion 62. The material can be preformed for the anchor wings 58 to be biased to an outwardly flared position extending perpendicular to the longitudinal axis of the conduit portion 62. The anchor wings 58 and triangular portion 60 are arcuate portions of a cylinder bending around an axis perpendicular to the longitudinal axis of the conduit portion 62.

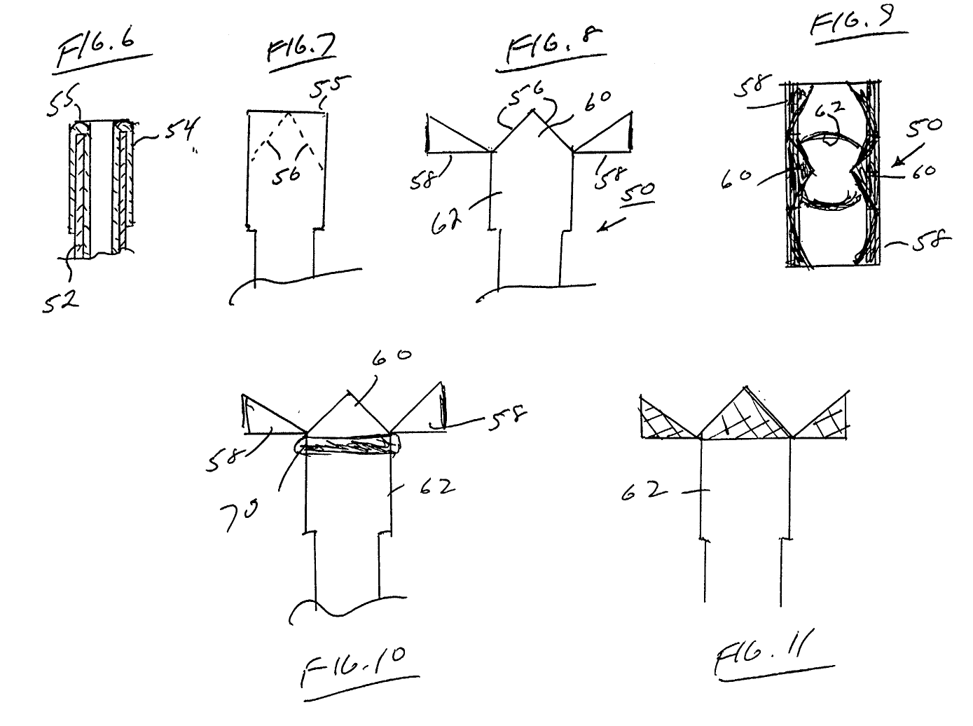

To attach the member, an incision IN is formed in the artery CA. The free end 55 is placed in the vessel CA and the wings 58 flare outwardly capturing the tube in the artery. A sewing cuff 70 (FIG. 10) may be provided on the tube 62 for stitching to the artery to prevent leakage. Also, a bio-glue may be provided at the incision IN to prevent leaks.

With the embodiment described, ePTFE only is exposed to blood flow. As an alternative, the wings 58 could be formed of open cell mesh material (e.g., nitinol, stainless steel, etc.) (FIG. 11) and left exposed for promoting tissue in-growth similar to that of open cell stents.

Having disclosed the present invention in a preferred embodiment, it will be appreciated that modifications and equivalents may occur to one of ordinary skill in the art having the benefits of the teachings of the present invention. It is intended that such modifications shall be included within the scope of the claims are appended hereto.

What is claimed is:

1. An apparatus for use in a coronary artery bypass procedure at a coronary vessel disposed lying at an exterior of a heart wall, the apparatus comprising;
a hollow rigid conduit adapted to be inserted into and retained within the heart wall of a heart chamber containing oxygenated blood with the conduit in blood-flow communication with blood contained within the chamber, said rigid conduit being sufficiently rigid to withstand collapsing in response to contraction forces of said heart wall;
a synthetic flexible conduit having a first end secured to said conduit for blood flow from said chamber to flow into said flexible conduit, said flexible conduit having a surface compatible to blood flow;
said flexible conduit having a second end secured to the coronary vessel with an opening of the second end in blood flow communication with a lumen of the coronary vessel;
the rigid conduit and flexible conduit defining a blood flow path between the openings of the first and second ends;
the flexible conduit bonded to and wrapped around said rigid conduit for said blood flow path to be defined by uninterrupted surface of said flexible conduit.

2. An apparatus according to claim 1 wherein:
said rigid conduit is formed of a rigid plastic material and said flexible conduit is formed of expanded polytetrafluoroethylene bonded to said rigid conduit by heating said rigid conduit for material of said rigid conduit to flow into micro-pores of said polytetrafluoroethylene.

3. An apparatus according to claim 1 comprising:
a plurality of reinforcing members secured to an outer surface of said flexible conduit along a length of said flexible conduit.

4. An apparatus according to claim 3 wherein:
said reinforcing members are a plurality of discrete rigid rings.

5. An apparatus according to claim 2 wherein:
said rigid plastic material is low density polyethylene having a melting point lower than that of said polytetrafluoroethylene.
